SEQUENCE LISTING (14 sequences)

SEQ ID NO: 1 - protein, 120 aa, synthetic construct
EVQLVETGGGLVQPKGSLKLSCAASGFSFNNNAMNWVRQAPGKGLEWVARIRSKTNNYEIYYAESVKDRFTISRDDSQSMLYLQMNNLKTDDTAMYYCVRDYNHVGFVYWGQGTQVTVST

SEQ ID NO: 2 - protein, 113 aa, synthetic construct
DIVMTQTTPSVPVTPGESVSISCRSSKSLLYSNGNTYLYWFLQRPGQSPQLLIYRMSNLASGVPDRFSGSGSGTAFTLRISRVEAEDVGVYYCMQHLEYPFTFGAGTKLELKR

SEQ ID NO: 3 - protein, 116 aa, synthetic construct
EVQLVESDGGFLQPGRSLKLSCAASGFTFSDYYMAWVRQAPTKGLEWVATISSDGSNTYYRDSVKGRFTISRDNAKTTLYLQMDSLRSEDTATYYCAGQAAGFASWGQGTLVTVSS

SEQ ID NO: 4 - protein, 108 aa, synthetic construct
DIQMTQSPSFLSASVGDRVTINCKASQNINKYLNWYQQKLGEAPKRLIYNTNNLQPGIPSRFSGSGSGTDYTLTISSLQPEDFATYFCLHHNSFPLTFGSGTKLEIKR

SEQ ID NO: 5 - protein, 709 aa, synthetic construct
EVQLVESDGGFLQPGRSLKLSCAASGFTFSDYYMAWVRQAPTKGLEWVATISSDGSNTYYRDSVKGRFTISRDNAKTTLYLQMDSLRSEDTATYYCAGQAAGFASWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGSGGGGSGGGGSEVQLVETGGGLVQPKGSLKLSCAASGFSFNNNAMNWVRQAPGKGLEWVARIRSKTNNYEIYYAESVKDRFTISRDDSQSMLYLQMNNLKTDDTAMYYCVRDYNHVGFVYWGQGTQVTVSTGGGGSGGGGSGGGGSDIVMTQTTPSVPVTPGESVSISCRSSKSLLYSNGNTYLYWFLQRPGQSPQLLIYRMSNLASGVPDRFSGSGSGTAFTLRISRVEAEDVGVYYCMQHLEYPFTFGAGTKLELKR

SEQ ID NO: 6 - protein, 108 aa, synthetic construct; identical to SEQ ID NO: 4

SEQ ID NO: 7 - protein, 725 aa, synthetic construct
QIVESGGGLVQPKESLKISCTASGFTFSNAAIYWVRQTPGKGLEWVGRIRTRPSKYATDYADSVRGRFTISRDDSKSMVYLQMDNLRTEDTAMYYCTPRATEDVPFYWGQGVMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGSGGGGSGGGGSEVQLVETGGGLVQPKGSLKLSCAASGFSFNNNAMNWVRQAPGKGLEWVARIRSKTNNYEIYYAESVKDRFTISRDDSQSMLYLQMNNLKTDDTAMYYCVRDYNHVGFVYWGQGTQVTVSTGGGGSGGGGSGGGGSGGGGSGGGGSGGGGSDIVMTQTTPSVPVTPGESVSISCRSSKSLLYSNGNTYLYWFLQRPGQSPQLLIYRMSNLASGVPDRFSGSGSGTAFTLRISRVEAEDVGVYYCMQHLEYPFTFGAGTKLELK

SEQ ID NO: 8 - protein, 220 aa, synthetic construct
DIVMTQTPSSQAVSAGEKVTMNCKSSQSVLYDENKKNYLAWYQQKSGQSPKLLIYWASTGESGVPDRFIGSGSGTDFTLTISSVQAEDLAVYYCQQYYDFPPTFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 9 - protein, 725 aa, synthetic construct
EVQLVETGGGLVQPKGSLKLSCAASGFSFNNNAMNWVRQAPGKGLEWVARIRSKTNNYEIYYAESVKDRFTISRDDSQSMLYLQMNNLKTDDTAMYYCVRDYNHVGFVYWGQGTQVTVSTGGGGSGGGGSGGGGSGGGGSGGGGSGGGGSDIVMTQTTPSVPVTPGESVSISCRSSKSLLYSNGNTYLYWFLQRPGQSPQLLIYRMSNLASGVPDRFSGSGSGTAFTLRISRVEAEDVGVYYCMQHLEYPFTFGAGTKLELKGGGGSGGGGSGGGGSQIVESGGGLVQPKESLKISCTASGFTFSNAAIYWVRQTPGKGLEWVGRIRTRPSKYATDYADSVRGRFTISRDDSKSMVYLQMDNLRTEDTAMYYCTPRATEDVPFYWGQGVMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 10 - protein, 220 aa, synthetic construct; identical to SEQ ID NO: 8

SEQ ID NO: 11 - protein, 725 aa, synthetic construct
QIVESGGGLVQPKESLKISCTASGFTFSNAAIYWVRQTPGKGLEWVGRIRTRPSKYATDYADSVRGRFTISRDDSKSMVYLQMDNLRTEDTAMYYCTPRATEDVPFYWGQGVMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGSGGGGSGGGGSDIVMTQTTPSVPVTPGESVSISCRSSKSLLYSNGNTYLYWFLQRPGQSPQLLIYRMSNLASGVPDRFSGSGSGTAFTLRISRVEAEDVGVYYCMQHLEYPFTFGAGTKLELKGGGGSGGGGSGGGGSGGGGSGGGGSGGGGSEVQLVETGGGLVQPKGSLKLSCAASGFSFNNNAMNWVRQAPGKGLEWVARIRSKTNNYEIYYAESVKDRFTISRDDSQSMLYLQMNNLKTDDTAMYYCVRDYNHVGFVYWGQGTQVTVST

SEQ ID NO: 12 - protein, 220 aa, synthetic construct; identical to SEQ ID NO: 8

SEQ ID NO: 13 - protein, 725 aa, synthetic construct
DIVMTQTTPSVPVTPGESVSISCRSSKSLLYSNGNTYLYWFLQRPGQSPQLLIYRMSNLASGVPDRFSGSGSGTAFTLRISRVEAEDVGVYYCMQHLEYPFTFGAGTKLELKGGGGSGGGGSGGGGSGGGGSGGGGSGGGGSEVQLVETGGGLVQPKGSLKLSCAASGFSFNNNAMNWVRQAPGKGLEWVARIRSKTNNYEIYYAESVKDRFTISRDDSQSMLYLQMNNLKTDDTAMYYCVRDYNHVGFVYWGQGTQVTVSTGGGGSGGGGSGGGGSQIVESGGGLVQPKESLKISCTASGFTFSNAAIYWVRQTPGKGLEWVGRIRTRPSKYATDYADSVRGRFTISRDDSKSMVYLQMDNLRTEDTAMYYCTPRATEDVPFYWGQGVMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 14 - protein, 220 aa, synthetic construct; identical to SEQ ID NO: 8

(19) United States
(12) Patent Application Publication
(10) Pub. No.: US 2025/0320299 A1
(43) Pub. Date: Oct. 16, 2025

(54) BISPECIFIC DENDRITIC CELL ENGAGER AND USES THEREOF

(71) Applicant: OBI PHARMA, INC., Taipei City (TW)

(72) Inventors: Ming-Tain Lai, Taipei City (TW); Jiann-Shiun Lai, Taipei City (TW); Yi-Chien Tsai, Taipei City (TW); Jing-Rong Huang, Taipei City (TW); Woan Eng Chan, Taipei City (TW); Ya-Chi Chen, Taipei City (TW)

(73) Assignee: OBI PHARMA, INC., Taipei City (TW)

(21) Appl. No.: 19/253,986

(22) Filed: Jun. 30, 2025

Related U.S. Application Data
(63) Continuation-in-part of application No. 18/685,917, filed on Feb. 23, 2024.
(60) Provisional application No. 63/238,229, filed on Aug. 30, 2021.

(51) Int. Cl. C07K 16/28; A61K 31/555; A61K 39/00; A61K 39/395; A61K 47/68; A61P 35/00; C07K 16/18

(52) U.S. Cl. CPC C07K 16/2851; A61K 31/555; A61K 39/39558; A61K 47/68037; A61K 47/6851; A61P 35/00; C07K 16/18; A61K 2039/505; C07K 2317/31; C07K 2317/73; C07K 2317/92

(57) ABSTRACT

The present disclosure relates to treating cancer patients with anti-dendritic cells (anti-DCs)/anti-immunogenic cell death (anti-ICD) bispecific engager combined with tumor ICD inducers to enhance the dendritic cell activity. Exemplary polyvalent proteins include at least one DC binding site and at least one ICD binding site. In certain embodiments, the binding sites may be linked through a constant immunoglobulin region. Anti-DC and anti-ICD monoclonal antibodies are also provided.

### CROSS-REFERENCE TO RELATED APPLICATIONS

This application is a continuation-in-part of U.S. patent application Ser. No. 18/685,917 filed Feb. 23, 2024, now pending. The prior application Ser. No. 18/685,917 is a 371 application of the international PCT application serial no. PCT/US2022/075612, filed Aug. 30, 2022, which claims the priority of Provisional Application No. 63/238,229, filed on Aug. 30, 2021. The entirety of each of the above mentioned patent applications is hereby incorporated by reference herein and made a part of this specification.

### FIELD

The present disclosure relates to a bispecific dendritic cell engager targeting dendritic cells (DCs) and immunogenic cell death (ICD) markers on tumor cells. Anti-DC/anti-ICD bispecific engagers are administered to enhance the dendritic cells activation and maturation with phagocytosis of ICD expressed tumor cells, so as to further induce tumor-specific cellular and humoral immunity. The disclosure provides methods for treating cancers using anti-DC/anti-ICD bispecific engager.

### SEQUENCE LISTING

The instant application contains a Sequence Listing XML, which has been submitted electronically in ASCII format and is hereby incorporated by reference in its entirety. Said ASCII copy, created Jun. 24, 2025, is named “155982-US-PA.xml” and is 17,908 bytes in size.

### BACKGROUND

The idea of using a bispecific antibody (BsAb) to efficiently retarget effector immune cells toward tumor cells emerged in the 1980s. Bispecific scaffolds are generally classified into two major groups with different pharmacokinetic properties, based on the absence or presence of an Fc fragment, IgG-like molecules and small recombinant bispecific formats, most of them deriving from single chain variable fragment (scFv).

Dendritic cells (DCs) are highly specialized antigen-presenting cells (APCs) with a unique ability to initiate the development of adaptive immune response when pulsed with antigens (Steinman, 1991). It is an efficient stimulator of B and T lymphocytes. While DCs catch up antigens, the antigens are processed into peptides for presentation on MHC class I molecules to CD8⁺ T-cells or presentation on MHC class II molecules to the CD4⁺ T-cells. Besides, cytokines secreted by the CD4⁺ T-cells could assist the maturation of B cells and the activation of cytotoxic T cells. Moreover, activated DCs also could secrete IL-12, IL-15, and type I IFNs to activate NK cells (Münz et al., 2005). Also, a high density of tumor-infiltrating DCs was found to be a better prognosis marker of clinical outcome (Dieu-Nosjean et al., 2008) with increased T cell activation (Ladányi et al., 2007). Antigen targeting DCs via CLEC9A could strongly enhance anti-tumor immunity. Other evidence also showed that antigen targeting CLEC9A could enhance immune responses of CD4⁺ T-cell, CD8⁺ T-cell, and B cell (Park H Y et al., 2013). Further study indicated that the CLEC9A could specifically recognize F-actin, a central component of the cellular cytoskeleton exposed by necrotic cells, and initiate cross-priming of DCs to CD8⁺ T-cell response against dead cell-associated antigens (Zhang J G et al., 2013). These results suggested that CLEC9A is a DC-restricted marker sensing damaged cells and antigens. Thus targeting CLEC9A⁺ DC can promote humoral and cellular immunity. Because of the central role of DCs in initiating immune responses, they would serve as an ideal target for boosting endogenous anti-tumor responses to eradicate tumors.

Immunogenic cell death (ICD) is defined by exposing damage-associated molecular patterns (DAMPs) from tumors in the tumor microenvironment (TME) which stimulates the host immune system. The ICD can be induced by chemotherapy, nanopulse stimulation, encapsulated nanoparticle, near-infrared photoimmunotherapy, and immune attacks (Zhou et al., 2019). Inducing ICD in tumors upregulates the expression of endogenous danger signals such as adenosine triphosphate (ATP), heat shock proteins (Hsp), calreticulin (CRT), and high-mobility group box1 protein (HMGB1) (Krysko et al., 2012). After engulfing immunogenic dead tumor cells by DCs, tumor antigen would be presented, followed by activating the tumor-specific cytotoxic T cell responses (Obeid et al., 2007). Notably, the induction of tumor ICD is associated with maintaining the long-lasting protective anti-tumor immunity (Zhou et al., 2019). Furthermore, the elevated CRT expression is a strong predictive marker for OS of cancer patients (Fucikova et al., 2016).

For successfully inducing the anti-tumor T cell activity by DCs, it requires three signals, which include capturing and presenting tumor-associated antigens (TAAs) on MHC molecules (signal 1), providing co-stimulation (signal 2) and soluble factors (signal 3) (Palucka and Banchereau, 2012; Palucka et al., 2011). Failure in signals 1, 2, and/or 3, which may modulate by tumors impairs the DC-mediated cross-presentation of tumor antigens and often induces T cell tolerance (Gabrilovich et al., 1997). DCs can be stimulated by several agonists through toll-like receptors (Schreibelt et al., 2010), STING (Ishikawa and Barber, 2008) and CD40 (O'Sullivan and Thomas, 2002) pathways. These agonists are either approved by US regulatory agencies or under clinical evaluation (Hübbe et al., 2020). Therefore, inducing tumor ICD followed by enhancing DC phagocytosis and activation is an attractive approach to boost the host's anti-tumor immunity and create immunological memory to eradicate tumor cells. The invention describes how to engage between the DCs and ICD tumors to enhance the host anti-tumor immunity.

### SUMMARY OF THE INVENTION

The present disclosure relates to an antibody or an antigen-binding fragment thereof, which includes a domain binding to an immunogenic cell death (ICD) marker on a tumor cell.

In certain embodiments, the ICD marker comprises calreticulin, heat shock protein (HSP), or other proteins exposed on the tumor cell surface during ICD.

In certain embodiments, the HSP is Hsp70 or Hsp90.

In certain embodiments, the ICD marker comprises calreticulin.

In certain embodiments, the present disclosure provides for an antibody or an antigen-binding fragment thereof further including a heavy chain variable domain having an amino acid sequence with at least about 90% sequence homology to SEQ ID NO: 1 and a light chain variable domain having an amino acid sequence with at least about 90% sequence homology to SEQ ID NO: 2.

In certain embodiments, the present disclosure provides for an antibody or an antigen-binding fragment, including a domain binding to a protein marker on a dendritic cell.

In certain embodiments, the protein marker includes CD1a, CD1c, CD11b, CD11c, CD16, CD32, CD103, CD115, CD123, CD207, CD301b, CD317, B220, BDCA1, BDCA2, BDCA3, BDCA4, CADM1, CCR2, CLEC9A, CXCR1, DCIR2, DEC205,EPCAM, Ly6C, SIRP, SiglecH or XCR1.

In certain embodiments, the protein marker is CLEC9A.

In certain embodiments, the present disclosure provides for an antibody or an antigen-binding fragment further including a heavy chain variable domain having an amino acid sequence with at least about 90% sequence homology to SEQ ID NO: 3 and a light chain variable domain having an amino acid sequence with at least about 90% sequence homology to SEQ ID NO: 4.

In certain embodiments, the present disclosure provides for an antibody or an antigen-binding fragment having a bispecific property and including an antibody or an antigen-binding fragment having a domain binding to an immunogenic cell death (ICD) marker on a tumor cell or a domain binding to a protein marker on a dendritic cell.

In certain embodiments, the present disclosure provides for an antibody or an antigen-binding fragment further including a first binding domain that specifically binds to calreticulin and a second binding domain that specifically binds to CLEC9A.

In certain embodiments, the present disclosure provides for an antibody or an antigen-binding fragment further including a heavy chain variable domain having an amino acid sequence with at least about 90% sequence homology to SEQ ID NO: 5 and a light chain variable domain having an amino acid sequence with at least about 90% sequence homology to SEQ ID NO: 6.

In certain embodiments, an isotype of the antibody is IgG, IgE, IgM, IgD, or IgA.

In certain embodiments, the antibody is an IgG antibody.

In certain embodiments, the IgG antibody is an lgG1, lgG2, lgG3, or lgG4 antibody.

In certain embodiments, the antibody is a human antibody.

In certain embodiments, the present disclosure provides for a pharmaceutical composition including the antibody or the antigen-binding fragment thereof as previously described and a pharmaceutically acceptable carrier.

The present disclosure also relates to a method for treating a patient with cancer, and the method includes a step of administering an effective amount of the pharmaceutical composition to a patient in need.

In certain embodiments, the cancer is ICD marker expressing cancer.

In certain embodiments, the ICD marker expressing cancer is selected from the group consisting of sarcoma, skin cancer, leukemia, lymphoma, brain cancer, glioblastoma, lung cancer, breast cancer, oral cancer, head-and-neck cancer, nasopharyngeal cancer, esophagus cancer, stomach cancer, liver cancer, bile duct cancer, gallbladder cancer, bladder cancer, pancreatic cancer, intestinal cancer, colorectal cancer, kidney cancer, cervix cancer, endometrial cancer, ovarian cancer, testicular cancer, buccal cancer, oropharyngeal cancer, laryngeal cancer, and prostate cancer.

In certain embodiments, the present disclosure provides for an antibody or an antigen-binding fragment thereof further including a heavy chain variable domain having an amino acid sequence with at least about 90% sequence homology to SEQ ID NO: 7 and a light chain variable domain having an amino acid sequence with at least about 90% sequence homology to SEQ ID NO: 8.

In certain embodiments, the present disclosure provides for an antibody or an antigen-binding fragment thereof further including a heavy chain variable domain having an amino acid sequence with at least about 90% sequence homology to SEQ ID NO: 9 and a light chain variable domain having an amino acid sequence with at least about 90% sequence homology to SEQ ID NO: 10.

In certain embodiments, the present disclosure provides for an antibody or an antigen-binding fragment thereof further including a heavy chain variable domain having an amino acid sequence with at least about 90% sequence homology to SEQ ID NO: 11 and a light chain variable domain having an amino acid sequence with at least about 90% sequence homology to SEQ ID NO: 12.

In certain embodiments, the present disclosure provides for an antibody or an antigen-binding fragment thereof further including a heavy chain variable domain having an amino acid sequence with at least about 90% sequence homology to SEQ ID NO: 13 and a light chain variable domain having an amino acid sequence with at least about 90% sequence homology to SEQ ID NO: 14.

In a preferred embodiment the combination of the present invention comprises one anti-calreticulin and anti-Clec9A bispecific antibody or a fragment thereof and one immune checkpoint blockade. Specifically, the immune checkpoint blockade is an anti-PD1 antibody.

In a preferred embodiment the combination of the present invention comprises one anti-calreticulin and anti-Clec9A bispecific antibody or a fragment thereof and one antibody drug conjugate (ADC). Specifically, the ADC is an anti-TROP2 ADC (also termed as OBI-902) as described in PCT patent publication WO2025064733A1.

In a preferred embodiment the combination of the present invention comprises one anti-calreticulin and anti-Clec9A bispecific antibody or a fragment thereof and one chemotherapy drug. Specifically, the chemotherapy drug is oxaliplatin.

In certain embodiments, the immune checkpoint is selected from the group consisting of PD-1/PD-L1 antigen, CTLA-4 (Cytotoxic T-lymphocyte-Associated Protein 4), LAG-3 (Lymphocyte Activation Gene 3), TIGIT (T-cell ImmunoGlobulin and Immunoreceptor Tyrosine-based inhibitory motif domain), Ceacam 1 (Carcinoembryonic antigen-related cell adhesion molecule 1), LAIR-1 (leucocyte-associated immunoglobulin-like receptor-1) or TIM-3 (T cell Immunoglobulin and Mucin domain-3).

### BRIEF DESCRIPTION OF THE DRAWINGS

A more complete understanding of the disclosure may be obtained by reference to the accompanying drawings when considered in conjunction with the subsequent detailed description. The embodiments illustrated in the drawings are intended only to exemplify the disclosure and should not be construed as limiting the disclosure to the illustrated embodiments.

FIG. 1 shows a schematic of the mechanism of action (MOA) of anti-DC/anti-ICD bispecific engager to boost the host anti-tumor immunity. Cancer patient is pre-treated with an ICD-inducing therapy, including target therapy, chemotherapy, radiotherapy, phototherapy, etc. After inducing ICD markers expressed on tumors, the patients treated with anti-DC/anti-ICD engager could help the phagocytosis of ICD expressed tumors by DCs following DC activation to initiate the cellular and humoral immunity. The engrafted tumor neoantigens are processed into peptides for presentation on MHC class I molecules to CD8⁺ T-cells or presentation on MHC class II molecules to the CD4⁺ T-cells. Cytokines secreted by the activated CD4⁺ T-cells could assist the maturation and activation of B and cytotoxic T cells to eradicate tumor cells. Enhancing the cancer-immunity cycle by anti-DC/anti-ICD bispecific engager will provide long-lasting benefits to patients via enhanced immunosurveillance of cancers.

FIGS. 2A to 2B show exposing endogenous danger signals to tumor cells. FIG. 2A shows exposing calreticulin (CRT) to tumor cells after treatment with Oxaliplatin. FIG. 2B shows exposing heat shock protein 70 (Hsp70) to tumor cells after treatment with Oxaliplatin.

FIG. 3 shows ATP release from tumor cells after treatment with Oxaliplatin.

FIG. 4 shows the CLEC9A expression on dendritic cells.

FIG. 5 shows enhance of DCs-mediated phagocytosis of tumor cells treated with Oxaliplatin.

FIG. 6 shows inhibition of activation markers expression on DCs cultured with tumor cells.

FIGS. 7A to 7B show increasing T-cell proliferation primed by DCs loaded with Oxaliplatin-treated tumor cells. FIG. 7A shows CD4⁺ T-cell. FIG. 7B shows CD8⁺ T-cell.

FIGS. 8A to 8B show the ELISA binding assay of Anti-CELC9A (EC10) and Anti-CRT (5B3-1) antibodies. FIG. 8A shows the EC₅₀ of EC10 to mouse CLEC9A-ECD binding. FIG. 8B shows the EC₅₀ of 5B3-1 to human CRT-ECD binding.

FIGS. 9A to 9B show the characterization of the antibody EC10 (α-mouse CLEC9A), 5B3-1 (α-CRT), and EC10 x 5B3-1 BsAb by cell binding assay. FIG. 9A shows the binding of EC10, 5B3-1, or EC10 x 5B3-1 BsAb on mouse CLEC9A overexpressed 293F cells. FIG. 9B shows the binding of EC10, 5B3-1, or EC10 x 5B3-1 BsAb on oxaliplatin-treated 4T1 cells.

FIGS. 10A to 10C show anti-tumor activity of EC10 x 5B3-1 BsAb combined with Oxaliplatin in a mouse 4T1 syngeneic model. FIG. 10A shows the schedule and treatment regimen. FIG. 10B shows the tumor growth curve. FIG. 10C shows photographs of dissected tumors and excised tumor weight.

FIGS. 11A to 11B show the cDC1 population and MHC-II expression of EC10 x 5B3-1 BsAb combined with Oxaliplatin in the mouse 4T1 syngeneic model. FIG. 11A shows the percentage of cDC1 (CD11c+/CLEC9A+). FIG. 11B shows the MFI of MHC-II on cDC1.

FIGS. 12A to 12C show CD4/CD8 T-cell and NK cell population of EC10 x 5B3-1 BsAb combined with Oxaliplatin in a mouse 4T1 syngeneic model. FIG. 12A shows the percentage of CD4 T cells. FIG. 12B shows the percentage of CD8 T-cells. FIG. 12C shows the percentage of NK cells.

FIGS. 13A to 13C show the tumor growth curves for EMT6, MC38, and KPC models, respectively.

FIGS. 14A to 14B show the tumor growth curves of CT26 (High TROP2 expression) colon cancer models. FIG. 14A shows the combination synergy effect by using immune checkpoint blockade (anti-PD1). FIG. 14B shows the combination synergy effect by using OBI-902 ADC.

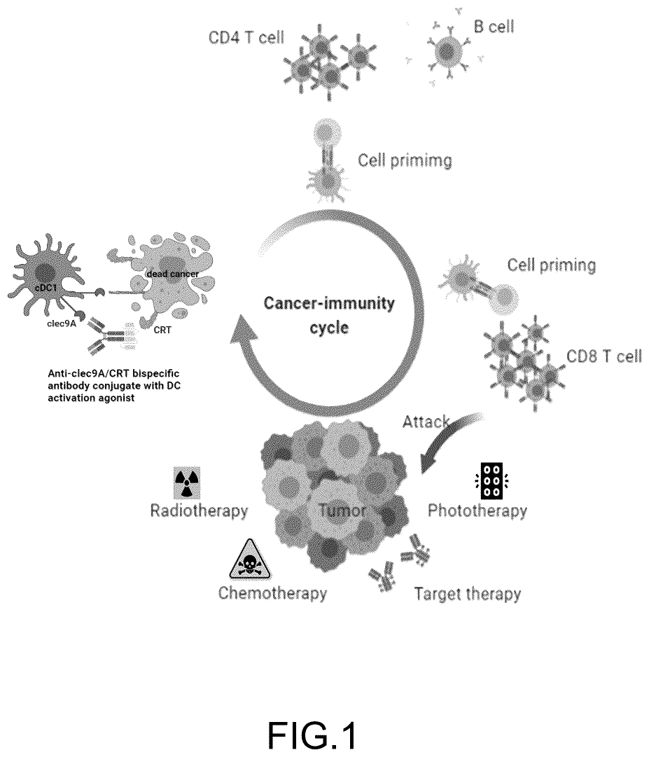

### DETAILED DESCRIPTION

As used herein, the articles “a” and “an” refer to one or more than one (i.e., at least one) of the grammatical object of the article. By way of example, “an element” means one element or more than one element.

The antibodies can be full-length or can comprise a fragment (or fragments) of the antibody having an antigen-binding portion, including, but not limited to, Fab, F(ab′)₂, Fab′, F(ab)′, Fv, single chain Fv (scFv), bivalent scFv (bi-scFv), trivalent scFv (tri-scFv), Fd, dAb fragment (Ward et al., (1989) Nature, 341:544-546), an isolated CDR, diabodies, triabodies, tetrabodies, linear antibodies, single-chain antibody molecules, and multispecific antibodies formed from antibody fragments. Single chain antibodies produced by joining antibody fragments using recombinant methods, or a synthetic linker, are also encompassed by the present invention (Bird et al., (1988) Science, 242:423-426; Huston et al., (1988) PNAS, 85:5879-5883).

All antibody isotypes are encompassed by the present invention, including IgG (e.g., IgG1, IgG2, IgG3, IgG4), IgM, IgA (IgA1, IgA2), IgD or IgE (all classes and subclasses are encompassed by the present invention). The antibodies or antigen-binding portions thereof may be mammalian (e.g., mouse, human) antibodies or antigen-binding portions thereof. The light chains of the antibody may be of kappa or lambda type.

In one embodiment, the present antibodies, or antigen-binding portions thereof, comprise at least one heavy chain variable region and/or at least one light chain variable region.

The present disclosure relates to anti-DCs/anti-ICD bispecific engager combined with tumor ICD inducers to treat cancer patients.

Accordingly, the present disclosure is based on the discovery that to elevate the dendritic cells with phagocytosis of ICD expressed tumor cells by anti-DCs/anti-ICD bispecific engager following the enhancement of DC activation and maturation. Targets for DCs include, but are not limited to, CD1a, CD1c, CD11b, CD11c, CD16, CD32, CD103, CD115, CD123, CD207, CD301b, CD317, B220, BDCA1, BDCA2, BDCA3, BDCA4, CADM1, CCR2, CLEC9A, CXCR1, DCIR2, DEC205, EPCAM, Ly6C, SIRP, SiglecH and XCR1.

Targets for ICD markers include, but are not limited to calreticulin, Hsp70, Hsp90, and the proteins expressed on cell membrane during tumor immunogenic cell death.

Cancers expressing ICD markers, but are not limited to, sarcoma, skin cancer, leukemia, lymphoma, brain cancer, glioblastoma, lung cancer, breast cancer, oral cancer, head-and-neck cancer, nasopharyngeal cancer, esophagus cancer, stomach cancer, liver cancer, bile duct cancer, gallbladder cancer, bladder cancer, pancreatic cancer, intestinal cancer, colorectal cancer, kidney cancer, cervix cancer, endometrial cancer, ovarian cancer, testicular cancer, buccal cancer, oropharyngeal cancer, laryngeal cancer, and prostate cancer.

The term “subject” can refer to a vertebrate having cancer or to a vertebrate deemed to be in need of cancer treatment. Subjects include all warm-blooded animals, such as mammals, such as a primate, and, more preferably, a human. Non-human primates are subjects as well. The term subject includes domesticated animals, such as cats, dogs, etc., livestock (for example, cattle, horses, pigs, sheep, goats, etc.) and laboratory animals (for example, mouse, rabbit, rat, gerbil, guinea pig, etc.). Thus, veterinary uses and medical formulations are contemplated herein.

An “effective amount,” as used herein, refers to a dose of the pharmaceutical composition that is sufficient to reduce the symptoms and signs of cancer, such as weight loss, pain and palpable mass, which is detectable, either clinically as a palpable mass or radiologically through various imaging means. The term “effective amount” and “therapeutically effective amount” are used interchangeably.

An exemplary, non-limiting range for a therapeutically or prophylactically effective amount of an antibody or antigen-binding portion of the invention is from about 0.05 μg/kg to about 500 mg/kg body weight, about 0.1 μg/kg to about 100 mg/kg body weight, about 1.0 μg/kg to about 10 mg/kg body weight, about 10 μg/kg to about 1.0 mg/kg body weight.

In certain embodiments, the antibodies or antigen-binding portions thereof include a variable light chain region comprising an amino acid sequence that is at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or about 100% homologous to SEQ ID NOs: 1-14.

### EXAMPLES

### Example 1: Exposure of Endogenous Danger Signals to Tumor Cell

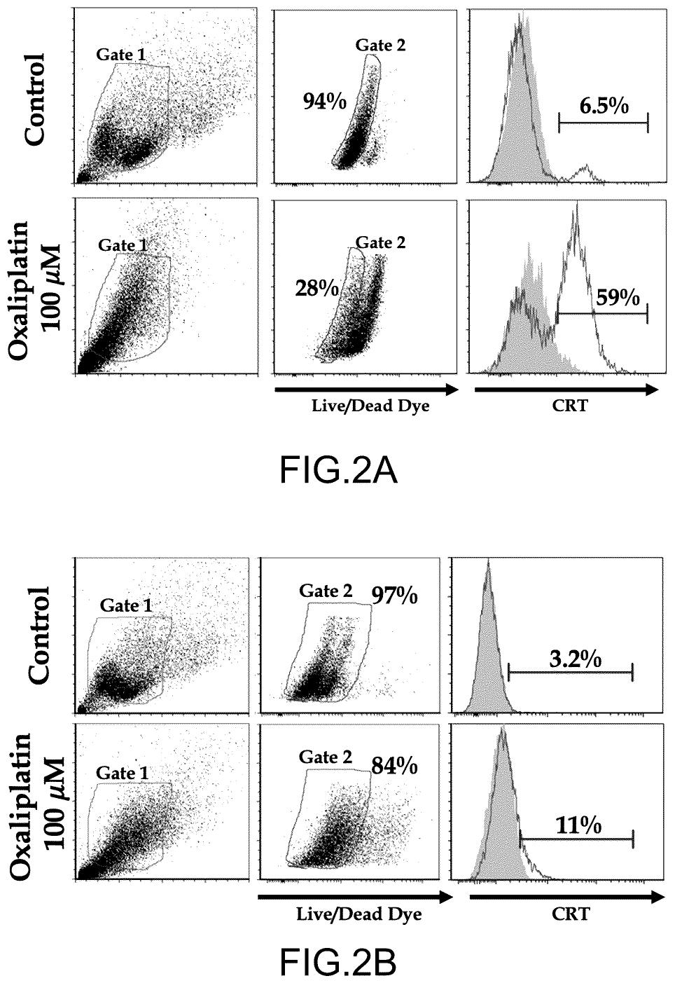

Mouse 4T1 breast cancer cells were seeded overnight in a 6-well culture plate. After cultivating overnight, the cells were treated or not with 100 μM Oxaliplatin for two days. Cells were collected and centrifuged, then stained with anti-CRT-Alexa 647 (Abcam, Cat #0080-012-310) and live/dead violet dye (ThermoFisher, Cat #L34964) at 4° C. for 30 minutes. Cells were washed and centrifuged. Surface expression of CRT and Hsp 70 on live 4T1 cells was analyzed by BD FACSCanto™ Clinical Flow Cytometry System (FACS CANTOII, BD Biosciences). FIG. 2A shows that CRT was translocated to the cell surface after treating Oxaliplatin. FIG. 2B shows that Hsp70 was translocated to the cell surface after treating Oxaliplatin. These results could demonstrate upregulation of surface CRT (from 6.5% to 59%) and Hsp70 (from 3.2% to 11%) after treating 100 μM Oxaliplatin.

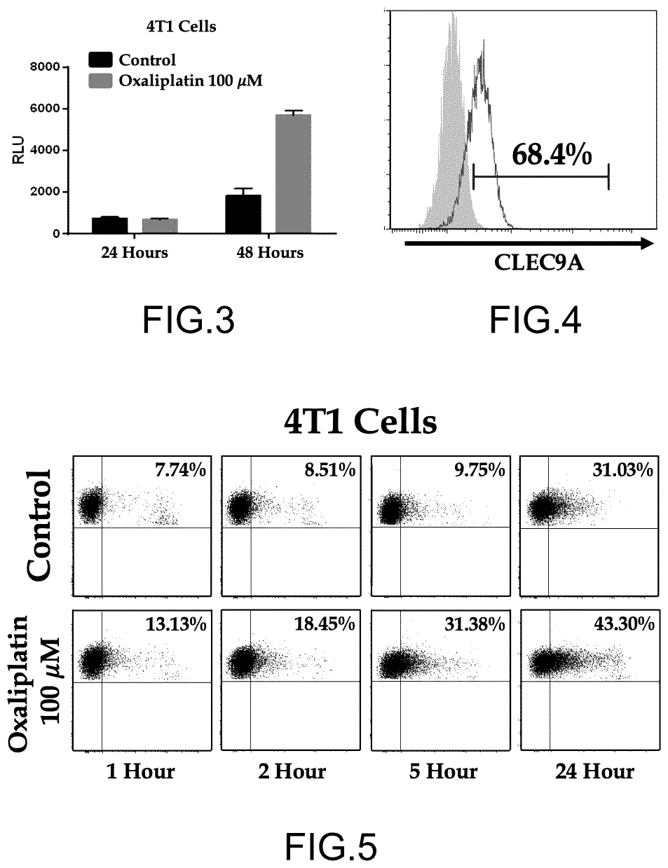

Furthermore, supernatants were collected and centrifuged then the ATP in the supernatant was determined by CellTiter-Glo® Luminescent Assay (Promega, Cat #G7570). Briefly, an equal volume of the collected supernatants and reagent were mixed and incubated in the dark for 10 minutes at room temperature. The luminescence was determined by a luminometer (MolecularDevice, SpectraMax L). FIG. 3 showed that ATP released to the medium was increased after treating 100 μM Oxaliplatin for two days.

### Example 2: Detection of CLEC9A Expression on Dendritic Cell

Mouse MutuDC 1940 cells were stained with monoclonal anti-CLEC9A antibody conjugated with PE fluorochrome (eBioscience, Cat #12-5975-82) at 4° C. for 30 minutes. Cells were then washed and collected. The binding of CLEC9A on MutuDC 1940 was analyzed by FACS CANTO II. FIG. 4 shows that CLEC9A was expressed (68.4%) on MutuDC 1940 cell.

### Example 3: Dendritic Cell-Mediated Phagocytosis of Tumor Cell

Mouse 4T1 breast cancer cells were labeled with CellTrace™ Far Red staining solution (ThermoFisher, Cat #C34564) at 37° C. for 30 minutes and then washed with PBS. Cells were seeded overnight in a 6-well plate. After cultivating overnight, cells were treated or not with 100 μM Oxaliplatin for two days. Then, cells were collected and counted. An equal number of MutuDC 1940 cells and 4T1 cells were mixed and incubated at 37° C. for the indicated time. After treatment, cells were collected and stained with anti-mouse CD11c-BV421 (Biolegend, Cat #117330) antibody at 4° C. for 30 minutes. Cells were washed and centrifuged, then analyzed by FACS CANTOII. 4T1 cell incorporation in MutuDC 1940 cells evaluated as the percentage of CD11c positive for far-red fluorescence incorporation. As FIG. 5 shows, 4T1 cells treated with Oxaliplatin increased DC-mediated phagocytosis of 4T1 cells by MutuDC 1940 cells (from 31.03% to 43.3%).

### Example 4: Inhibition of Activation Markers Expression on Dendritic Cell Co-Cultured With Tumor Cell

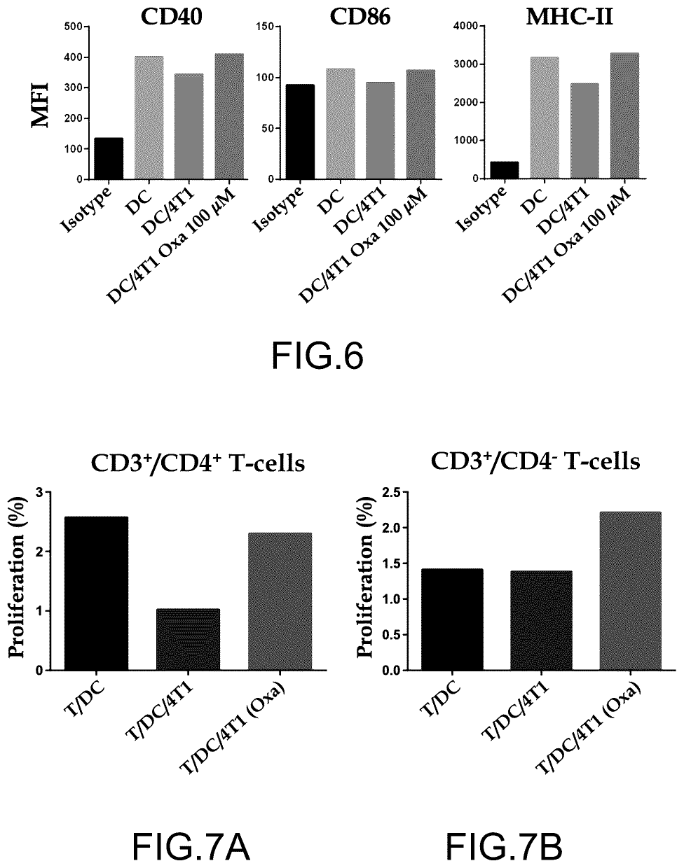

Mouse 4T1 breast cancer cells were seeded overnight in a 6-well plate. After cultivating overnight, cells were treated with or without 100 μM Oxaliplatin for two days. Then, cells were collected and counted. An equal number of MutuDC 1940 cells and 4T1 cells were mixed and incubated at 37° C. for 24 hours. After treatment, cells were collected and blocked by TruStainFcX™ PLUS antibody (anti-mouse CD16/32 antibody) (Biolegend, Cat #100512) at 4° C. for 10 minutes then stained with anti-mouse CD11c-BV421 (Biolegend, Cat #117330), anti-mouse CD40-PE (Biolegend, Cat #124610), anti-mouse IA/IE-APC/Cyanine7 (Biolegend, Cat #107628) and anti-mouse CD86-BV510 (Biolegend, Cat #105040) antibody at 4° C. for 30 minutes. Cells were washed and centrifuged, then analyzed by FACS CANTO II. FIG. 6 showed that CD40, CD86, and MHC-II expression were slightly decreased on MutuDC 1940 cell after culturing with 4T1 cell but not with Oxaliplatin treated 4T1 cell.

### Example 5: Increasing of T Cell Proliferation Primed by Dendritic Cell Loaded With Oxaliplatin Treated Tumor Cell

Mouse 4T1 breast cancer cells were seeded overnight in a 6-well plate. After cultivating overnight, cells were treated with or without 100 μM Oxaliplatin for two days. Then, cells were collected and counted. An equal number of MutuDC 1940 cells and 4T1 cells were mixed and incubated at 37° C. for 24 hours. Mouse Pan T cells were purified from splenocytes of C57BL/6 mouse using Pan T cell isolation kit II (Miltenyi Biotec, Cat #130-095-130) and then labeled with CellTrace™ Far Red staining solution (ThermoFisher, Cat #C34564) at 37° C. for 30 minutes. Cells were washed and counted. 5-fold Far Red labeled mouse Pan T cells were added to the co-cultured MutuDC 1940 cells and 4T1 cells for another 72 hours. Cells were collected and stained with anti-mouse CD3-FITC (Biolegend, Cat #100203) and anti-mouse CD4-PE (Biolegend, Cat #100407) at 4° C. for 30 minutes. Cells were washed and centrifuged, then analyzed by FACS CANTO II. CD4 T-cell proliferation was calculated by gating on CD3⁺/CD4⁺ cells and setting a marker that contained at least 97% of cells in unstimulated samples. FIG. 7A shows that the proliferation of CD4⁺T-cells was inhibited after culturing with dendritic cells coculture with 4T1 cells but not with Oxaliplatin treated 4T1 cells. Another CD8⁺T-cell proliferation was calculated by gating on CD3⁺/CD4⁻ cells and setting a marker that contained at least 98% of cells in unstimulated samples. FIG. 7B shows that the proliferation of CD8⁺T-cells was increased after culturing with dendritic cells coculture with Oxaliplatin treated 4T1 cells but not with control 4T1 cells.

### Example 6: ELISA Assay of the Protein Binding Activity of Antibodies, EC10 (α-Mouse CLEC9A) and 5B3-1 (α-CRT)

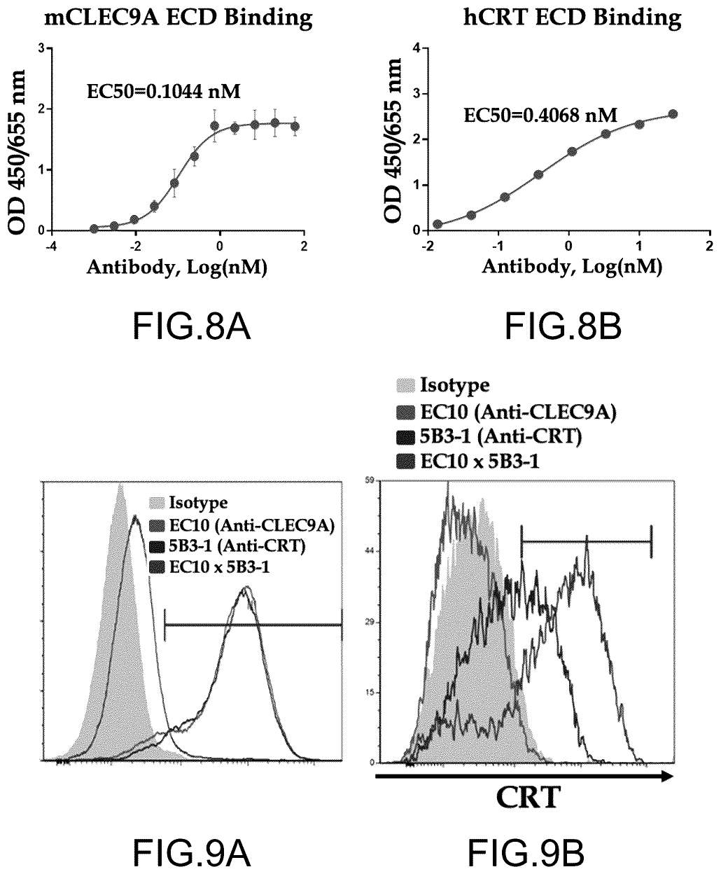

200 ng per well of mouse CLEC9A-ECD or human CRT-ECD proteins (OBI Pharma Inc., in-house produced) were coated in 96-well plates (Thermo Fisher, Cat #44-2402-21) at 4° C. overnight. The plates were washed with PBS containing 0.2% Tween-20 (PBST), then blocked with PBST containing 5% BSA for one hour at room temperature. Plates were washed with PBST and incubated with three-fold serial dilutions of the antibodies, α-mouse CLEC9A (OBI Pharma, Inc., named EC10) or α-CRT (OBI Pharma, Inc., named 5B3-1) for another two hours at room temperature. After washing, goat anti-mouse IgG (H+L) conjugated with HRP (JacksonImmunoResearch, Cat #115-035-062) was added and incubated for one hour at room temperature. The plates were washed and the reaction was developed with TMB substrates for 20 minutes at room temperature. The plates were then read on an ELISA reader (Molecular Devices, SpectraMax M2) at OD 450 nm. EC₅₀ of EC10 to mouse CLEC9A-ECD is 0.1044 nM as shown in FIG. 8A. EC₅₀ of 5B3-1 to human CRT-ECD is 0.4068 nM as shown in FIG. 8B.

### Example 7: Analysis of Cell Binding Activity of Antibodies, EC10 (α-mouse CLEC9A), 5B3-1 (α-CRT), and Bispecific Antibody (BsAb EC10 x 5B3-1)

Mouse CLEC9A overexpressed 293F or Oxaliplatin treated cells were stained with 30 nM, mouse IgG2a (Biolegend, Cat #400202), α-mouse CLEC9A (OBI Pharma, Inc., named EC10), α-CRT (OBI Pharma, Inc., named 5B3-1) or α-mouse CLEC9A x α-CRT (OBI Pharma, Inc., named EC10 x 5B3-1) bispecific antibody (BsAb) at 4° C. for 30 minutes. Samples were washed and centrifuged then stained with anti-mouse IgG labeled with FITC (SouthernBiotech, Cat #1032-02) at 4° C. for 30 minutes. Cells were then washed and collected. The binding activity of antibodies were analyzed by FACS CANTO II. FIG. 9A showed the binding activity of EC10 (55.1%) and EC10 x 5B3-1 BsAb (55.8%) on mouse CLEC9A overexpressed 293F cells. FIG. 9B showed the binding activity of 5B3-1 (41.2%) and EC10 x 5B3-1BsAb (77.6%) but not EC10 (1.9%) on oxaliplatin treated 4T1 cells.

### Example 8: Anti-Tumor Activity of EC10 x 5B3-1 BsAb Combined With Oxaliplatin in Mouse Syngeneic 4T1 Tumor Model

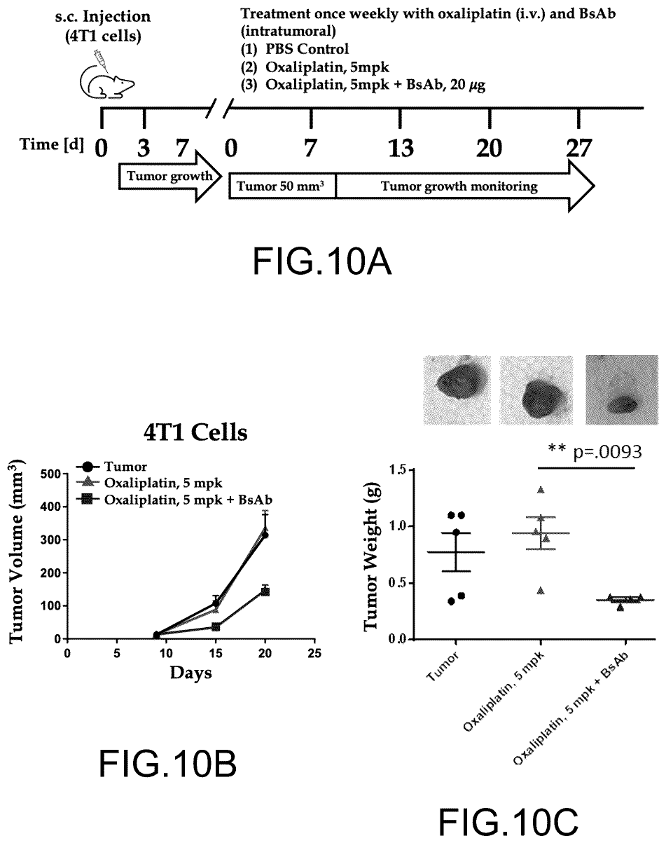

1×10⁵ 4T1 breast cancer cells was injected through subcutaneous injection (s.c. injection) on the right flank of mice in 100 μL sterile PBS. Five mice of each were randomized into three treatment groups on day 7 following tumor inoculation when the tumor size reached 50 mm³. PBS, Oxaliplatin (MedChemExpress, Cat #, HY-17371), and EC10 x 5B3-1 BsAb (OBI Pharma, Inc.) were administered on days 7, 13 and 20, intravenously or intratumorally (5 mg/kg for Oxaliplatin through intravenous and 20 μg for BsAb through intratumor). Tumor length, and width were recorded weekly. Tumor volumes were calculated using the formula: volume (mm³)=([width]²×length)/2. Results were shown as arithmetic meantstandard error of the mean (SEM). Statistical comparisons were performed by two-tailed Student's t-test. Significance set of probability “p” was set as the level at *p<0.05, **p<0.01, ***, p<0.001. FIG. 10A shows the schematic diagram for in vivo 4T1 tumor study in BALB/c mice. Mice were sacrificed at Day 27. FIG. 10B shows the tumor growth curve of 4T1 tumors. FIG. 10C shows the photographs and tumor weight of each group. It indicated that there was no difference between treating Oxaliplatin alone and control tumor group. However, treating EC10 x 5B3-1 BsAb and Oxaliplatin could induce a significant inhibition in 4T1 tumor growth and reduce tumor weight.

### Example 9: EC10 x 5B3-1 BsAb Combined With Oxaliplatin Enhanced the Immune Cell Population in Mouse Syngenic 4T1 Tumor Model

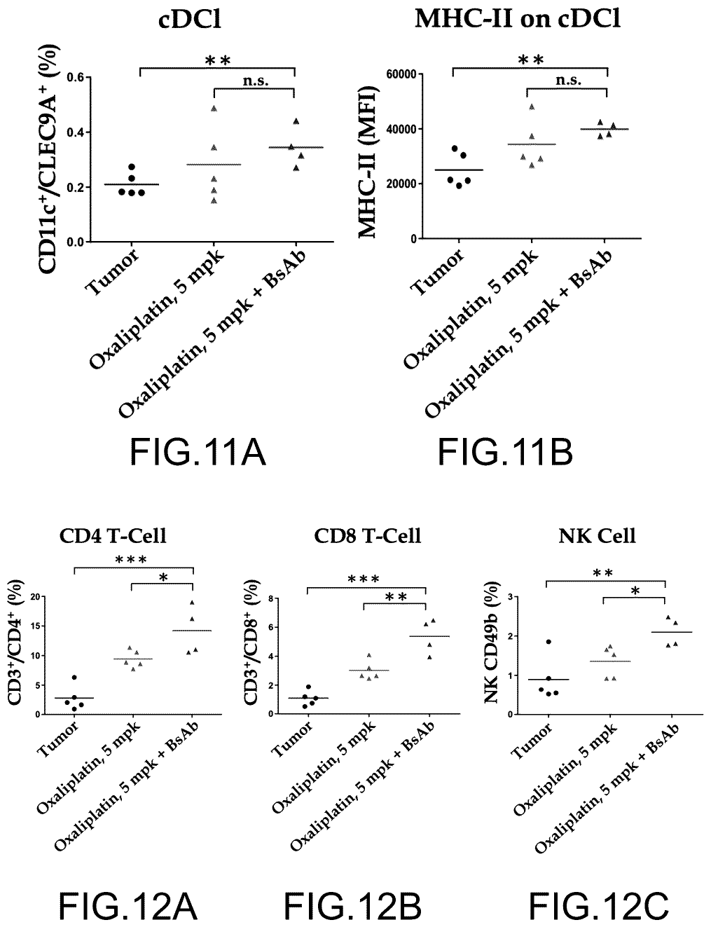

Splenocytes were collected from a mouse syngenic 4T1 breast cancer tumor model on the sacrificed day. 1×10⁶ cells of each sample were blocked by purified anti-mouse CD16/32 antibody (BioLegend, Cat #101302) at 4° C. for 10 minutes. Then stained with group A antibodies [α-mouse CD11c (Biolegend, Cat #117311), α-mouse CLEC9A (Biolegend, Cat #143504), and α-mouse I-A/I-E (Biolegend, Cat #107628)] and group B antibodies [α-mouse CD3 (Biolegend, Cat #100210), α-mouse CD4 (Biolegend, Cat #100210), α-mouse CD8 (Biolegend, Cat #100210), and α-mouse CD49b (Biolegend, Cat #100210)] at 4° C. for 30 minutes. Cells were washed and centrifuged, then analyzed by FACS CANTO II. cDC1 cell population was calculated by gating on CD11c⁺/CLEC9A⁺ cells and setting a marker that contained at least 97% of cells in isotype control samples. MHC-II on cDC1 was calculated by mean fluorescence intensity (MFI). CD4 T-cell population was calculated by CD3⁺/CD4⁺, CD8 T-cell was CD3+/CD8+and NK cell was CD3⁻/CD49b⁺. FIG. 11A shows that EC10 x 5B3-1 BsAb combined with Oxaliplatin could increase the most cDC1 population in the spleen. FIG. 11B also shows that EC10 x 5B3-1 BsAb combined with Oxaliplatin could increase the most MHC-II expression on cDC1. FIG. 11 shows that EC10 x 5B3-1 BsAb combined with Oxaliplatin could increase the CD4⁺T-cell (FIG. 12A), CD8⁺ T-cell (FIG. 12B), and NK cell (FIG. 12C) population in mouse spleen.

### Example 10: Anti-Tumor Activity of a Bispecific Antibody (EC10 x 5B3-1) in Syngeneic Mouse Tumor Models

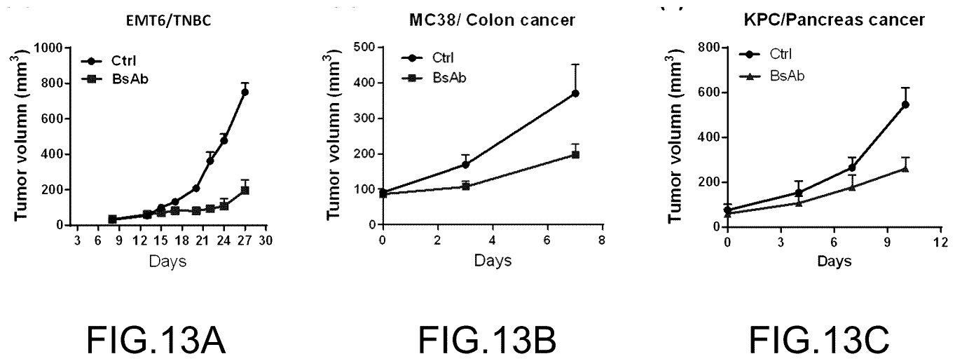

A total of 2×10⁵ EMT6 (triple-negative breast cancer), MC38 (colon cancer), or KPC (pancreatic cancer) cells were subcutaneously injected into the right flank of mice in a mixture of 50 μL sterile PBS and 50 μL Matrigel. On days 7 to 10 post-inoculation, when tumor volumes reached 50-100 mm³, mice were randomized into two treatment groups (n=5-8 per group). The bispecific antibody (EC10 x 5B3-1) was administered subcutaneously once per week at a dose of 20 μg. Tumor length and width were measured weekly, and tumor volumes were calculated using the formula: volume (mm³)=(width²×length)/2. Data was presented as the arithmetic mean±standard error of the mean (SEM). FIGS. 13A, 13B, and 13C showed the tumor growth curves for EMT6, MC38, and KPC models, respectively. Mice treated with bsAb exhibited significant tumor growth inhibition across all three cancer models, demonstrating the broad applicability of this bsAb without the need for target pre-screening.

### Example 11: Anti-Tumor Activity of Anti-CRT and Anti-CLEC9A Bispecific Antibodies (BsAbs) in Combination With an anti-PD1 Antibody or an Anti-TROP2 ADC (OBI-902) in a Syngeneic CT26 Mouse Tumor Model

### 11-1. Anti-PD1 Antibody Combination Assay

A total of 2×10⁵ CT26 cells were injected subcutaneously into the right flank of mice in a mixture of 50 μL sterile PBS and 50 μL matrix gel. On day 7 post-inoculation, when tumor volumes reached 50-100 mm³, mice were randomized into four treatment groups (n=5 per group). Treatments included PBS (control), α-PD-1 (BioXcell, Cat. No. BE0146), bispecific antibody (EC10 x 5B3-1) and the combination. α-PD-1 was administered intraperitoneal at 50 μg per dose, and BsAb was administered subcutaneously at 20 μg per dose. All treatments were given once weekly. Tumor length and width were measured weekly, and tumor volumes were calculated using the formula: volume (mm³)=(width²×length)/2. Data was presented as arithmetic mean±standard error of the mean (SEM). Statistical comparisons were performed using a two-tailed Student's t-test.

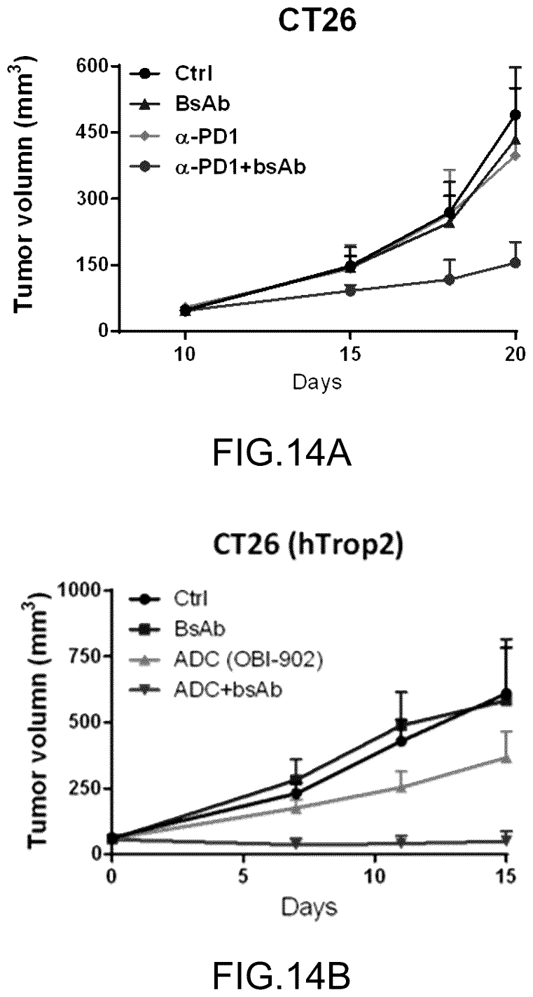

FIG. 14A showed the tumor growth curves of CT26 tumors. Neither BsAb nor α-PD-1 monotherapy significantly inhibited tumor growth compared to the control group. However, the combination of BsAb and α-PD-1 produced a significant synergistic effect (P=0.0106), resulting in marked suppression of CT26 tumor growth.

### 11-2. OBI-902 (Anti-TROP2 Antibody Drug Conjugate) Combination Assay

A total of 5×10⁵ CT26 (High TROP2 expression) cells were injected subcutaneously into the right flank of mice in a mixture of 50 μL sterile PBS and 50 μL matrix gel. On day 7 post-inoculation, when tumor volumes reached 50-100 mm³, mice were randomized into four treatment groups (n=8 per group): PBS (control), OBI-902, C5 bispecific antibody (10B4 x 5B3-1) and the combination. OBI-902 was administered intravenously at a dose of 120 μg, and BsAb was administered subcutaneously at a dose of 60 μg. All treatments were given once weekly. Tumor length and width were measured weekly, and tumor volumes were calculated using the formula: volume (mm³)=(width²×length)/2. Data was presented as arithmetic mean±standard error of the mean (SEM). Statistical comparisons were performed using a two-tailed Student's t-test.

FIG. 14B showed the tumor growth curves of CT26 tumors. BsAb monotherapy did not significantly inhibit tumor growth compared to the control group. OBI-902 alone demonstrated 40% tumor growth inhibition (TGI) on Day 15. Notably, the combination of BsAb and OBI-902 resulted in a pronounced synergistic effect, achieving 92% TGI on Day 15 (p=0.0012), indicating substantial suppression of CT26 (hTrop2) tumor growth.

### REFERENCES

1. Dieu-Nosjean, M.-C., Antoine, M., Danel, C., Heudes, D., Wislez, M., Poulot, V., Rabbe, N., Laurans, L., Tartour, E., de Chaisemartin, L., et al. (2008). Long-term survival for patients with non-small-cell lung cancer with intratumoral lymphoid structures. J. Clin. Oncol. 26, 4410-4417.
2. Fucikova, J., Becht, E., Iribarren, K., Goc, J., Remark, R., Damotte, D., Alifano, M., Devi, P., Biton, J., Germain, C., et al. (2016). Calreticulin Expression in Human Non-Small Cell Lung Cancers Correlates with Increased Accumulation of Antitumor Immune Cells and Favorable Prognosis. Cancer Res 76, 1746-1756.
3. Krysko, D.V., Garg, A.D., Kaczmarek, A., Krysko, O., Agostinis, P., and Vandenabeele, P. (2012). Immunogenic cell death and DAMPs in cancer therapy. Nat. Rev. Cancer 12, 860-875.
4. Ladányi, A., Kiss, J., Somlai, B., Gilde, K., Fejos, Z., Mohos, A., Gaudi, I., and Tímár, J. (2007). Density of DC-LAMP (+) mature dendritic cells in combination with activated T lymphocytes infiltrating primary cutaneous melanoma is a strong independent prognostic factor. Cancer Immunol. Immunother. 56, 1459-1469.
5. Münz, C., Dao, T., Ferlazzo, G., de Cos, M.A., Goodman, K., and Young, J.W. (2005). Mature myeloid dendritic cell subsets have distinct roles for activation and viability of circulating human natural killer cells. Blood 105, 266-273.
6. Obeid, M., Tesniere, A., Ghiringhelli, F., Fimia, G.M., Apetoh, L., Perfettini, J.-L., Castedo, M., Mignot, G., Panaretakis, T., Casares, N., et al. (2007). Calreticulin exposure dictates the immunogenicity of cancer cell death. Nat. Med. 13, 54-61.
7. Park HY, Light A, Lahoud MH, Caminschi I, Tarlinton DM and Shortman K. (2013). Evolution of B cell responses to Clec9Atargeted antigen. Journal of immunology 191, 4919-4925.
8. Schreibelt, G., Tel, J., Sliepen, K.H.E.W.J., Benitez-Ribas, D., Figdor, C.G., Adema, G.J., and de Vries, I.J.M. (2010). Toll-like receptor expression and function in human dendritic cell subsets: implications for dendritic cell-based anti-cancer immunotherapy. Cancer Immunol Immunother 59, 1573-1582.
9. Steinman, R.M. (1991). The dendritic cell system and its role in immunogenicity. Annu. Rev. Immunol. 9, 271-296.
10. Zhang JG, Czabotar PE, Policheni AN, Caminschi I, Wan SS, Kitsoulis S, Tullett KM, Robin AY, Brammananth R, van Delft MF, Lu J, O'Reilly LA, Josefsson EC, Kile BT, Chin WJ, Mintern JD, et al. (2012). The dendritic cell receptor Clec9A binds damaged cells via exposed actin filaments. Immunity. 36, 646-657.
11. Zhou, J., Wang, G., Chen, Y., Wang, H., Hua, Y., and Cai, Z. (2019). Immunogenic cell death in cancer therapy: Present and emerging inducers. J Cell Mol Med 23, 4854-4865.

Unless defined otherwise, all technical and scientific terms and any acronyms used herein have the same meanings as commonly understood by one of ordinary skill in the art in the field of this invention. Although any compositions, methods, kits, and means for communicating information similar or equivalent to those described herein can be used to practice this invention, the preferred compositions, methods, kits, and means for communicating information are described herein.

All references cited herein are incorporated herein by reference to the full extent allowed by law. The discussion of those references is intended merely to summarize the assertions made by their authors. No admission is made that any reference (or a portion of any reference) is relevant to the prior art. Applicants reserve the right to challenge the accuracy and pertinence of any cited reference.

1. A bispecific antibody or an antigen-binding fragment thereof, comprising:
a first domain binding to an immunogenic cell death (ICD) marker on a tumor cell, wherein the ICD marker comprises calreticulin or heat shock protein; and
a second domain binding to a protein marker on a dendritic cell, wherein the protein marker comprises CD1a, CD1c, CD11b, CD11c, CD16, CD32, CD103, CD115, CD123, CD207, CD301b, CD317, B220, BDCA1, BDCA2, BDCA3, BDCA4, CADM1, CCR2, CLEC9A, CXCR1, DCIR2, DEC205, EPCAM, Ly6C, SIRP, SiglecH or XCR1.

2. The bispecific antibody or the antigen-binding fragment thereof of claim 1, further comprising:
a heavy chain variable domain comprising an amino acid sequence with at least 90% sequence homology to SEQ ID NO: 7; and
a light chain variable domain comprising an amino acid sequence with at least 90% sequence homology to SEQ ID NO: 8.

3. The bispecific antibody or the antigen-binding fragment thereof of claim 1, further comprising:
a heavy chain variable domain comprising an amino acid sequence with at least 90% sequence homology to SEQ ID NO: 9; and
a light chain variable domain comprising an amino acid sequence with at least 90% sequence homology to SEQ ID NO: 10.

4. The bispecific antibody or the antigen-binding fragment thereof of claim 1, further comprising:
a heavy chain variable domain comprising an amino acid sequence with at least 90% sequence homology to SEQ ID NO: 11; and
a light chain variable domain comprising an amino acid sequence with at least 90% sequence homology to SEQ ID NO: 12.

5. The bispecific antibody or the antigen-binding fragment thereof of claim 1, further comprising:
a heavy chain variable domain comprising an amino acid sequence with at least 90% sequence homology to SEQ ID NO: 13; and
a light chain variable domain comprising an amino acid sequence with at least 90% sequence homology to SEQ ID NO: 14.

6. The bispecific antibody or the antigen-binding fragment thereof of claim 1, wherein the protein marker is CLEC9A.

7. The bispecific antibody or the antigen-binding fragment thereof of claim 1, wherein the ICD marker is calreticulin (CRT).

8. The bispecific antibody or an antigen-binding fragment thereof of claim 1, wherein an isotype of the antibody is IgG, IgE, IgM, IgD, or IgA.

9. The bispecific antibody or an antigen-binding fragment thereof of claim 8, wherein the IgG antibody is an lgG1, lgG2, lgG3, or lgG4 antibody.

10. A pharmaceutical composition, comprising:
the bispecific antibody or the antigen-binding fragment thereof as in any one of claims 1-5; and
a pharmaceutically acceptable carrier.

11. A method of treating an ICD marker expressing cancer in a patient, comprising:
administering an effective amount of the pharmaceutical composition of claim 10 to the patient in need;
wherein the effective amount comprises from 0.05 μg/kg to 500 mg/kg.

12. The method of claim 11, wherein the ICD marker expressing cancer is selected from the group consisting of sarcoma, skin cancer, leukemia, lymphoma, brain cancer, glioblastoma, lung cancer, breast cancer, oral cancer, head-and-neck cancer, nasopharyngeal cancer, esophagus cancer, stomach cancer, liver cancer, bile duct cancer, gallbladder cancer, bladder cancer, pancreatic cancer, intestinal cancer, colorectal cancer, kidney cancer, cervix cancer, endometrial cancer, ovarian cancer, testicular cancer, buccal cancer, oropharyngeal cancer, laryngeal cancer, and prostate cancer.

13. A method for treating cancer, wherein the method comprising administering to a patient in need thereof a therapeutically effective amount of a pharmaceutical composition comprising an anti-CRT and anti-CLEC9A bispecific antibody or the antigen-binding fragment thereof as in any one of claims 1-5, in combination with an anti-negative immune checkpoint antibody, an antibody drug conjugate (ADC) or a chemotherapy drug.

14. The method of claim 13, wherein the anti-negative immune checkpoint antibody is an anti-PD-1 or anti-PD-L1 antibody, the ADC is an anti-TROP2 ADC (OBI-902), and the chemotherapy drug is oxaliplatin.
